(19) United States
(12) Patent Application Publication
(10) Pub. No.: US 2012/0213663 A1
(43) Pub. Date: Aug. 23, 2012

(54) METHOD OF REMOVING E. COLI BACTERIA FROM AN AQUEOUS SOLUTION

(75) Inventors: MUATAZ ALI ATIEH, DHAHRAN (SA); AMJAD BAJES KHALIL, DHAHRAN (SA); TAHAR LAOUI, DHAHRAN (SA); SAMER MOHAMMED AL HAKAMI, DHAHRAN (SA)

(73) Assignee: KING FAHD UNIVERSITY OF PETROLEUM AND MINERALS, DHAHRAN (SA)

(21) Appl. No.: 13/033,462

(22) Filed: Feb. 23, 2011

(51) Int. Cl. A61L 2/12; A61L 2/16; B82Y 30/00

(52) U.S. Cl. 422/21; 422/28; 977/752; 977/773

(57) ABSTRACT

The method of removing Escherichia coli (E. coli) bacteria from an aqueous solution includes the step of mixing multi-walled carbon nanotubes into an aqueous solution containing E. coli bacteria. The multi-walled carbon nanotubes have an antimicrobial effect against the E. coli bacteria. The multi-walled carbon nanotubes may be mixed into the aqueous solution at a concentration of approximately 0.002 g of multi-walled carbon nanotubes per 100 ml of the aqueous solution. In order to enhance antimicrobial activity, the multi-walled carbon nanotubes in the solution may be treated with microwave radiation, thus generating heat to further destroy the bacteria. In order to further enhance antimicrobial activity, the multi-walled carbon nanotubes may be functionalized with a carboxylic (COOH) group, functionalized with a phenol (C₅H₅OH) group, functionalized with a C18 group, such as 1-octadecanol (C₁₈H₃₈O), or may be impregnated with silver nanoparticles.

### BACKGROUND OF THE INVENTION

1. Field of the Invention

The present invention relates generally to disinfection techniques and methods of treating water or aqueous solution for the removal of microorganisms therefrom, and particularly to a method of removing Escherichia coli (E. coli) bacteria from an aqueous solution.

2. Description of the Related Art

Escherichia coli (commonly abbreviated K coli) is a Gram-negative, rod-shaped bacterium that is commonly found in the lower intestine of warm-blooded organisms (endotherms). Most E. coli strains are harmless, but some, such as serotype O157:H7, can cause serious food poisoning in humans, and are occasionally responsible for product recalls. The harmless strains are part of the normal flora of the gut, and can benefit their hosts by producing vitamin K₂ and by preventing the establishment of pathogenic bacteria within the intestine.

Certain strains of E. coli, such as O157:H7, O121 and O104:H21, produce potentially lethal toxins. Food poisoning caused by E. coli is usually caused by eating unwashed vegetables or undercooked meat. O157:H7 is also notorious for causing serious and even life-threatening complications, such as haemolytic-uremic syndrome. This particular strain is linked to the 2006 United States E. coli outbreak due to fresh spinach. Severity of the illness varies considerably. It can be fatal, particularly to young children, the elderly, or the immunocompromised, but is more often mild.

If E. coli bacteria escape the intestinal tract through a perforation (for example from an ulcer, a ruptured appendix, or due to a surgical error) and enter the abdomen, they usually cause peritonitis that can be fatal without prompt treatment. However, E. coli are extremely sensitive to such antibiotics as streptomycin or gentamicin. This, however, could easily change, since E. coli quickly acquires drug resistance. Recent research suggests that treatment with antibiotics does not improve the outcome of the disease, and may, in fact, significantly increase the chance of developing haemolytic-uremic syndrome.

Intestinal mucosa-associated E. coli are also observed in increased numbers in the inflammatory bowel diseases, Crohn's disease, and ulcerative colitis. Invasive strains of E. coli exist in high numbers in the inflamed tissue, and the number of bacteria in the inflamed regions correlates to the severity of the bowel inflammation.

Resistance to beta-lactam antibiotics has become a particular problem in recent decades, as strains of bacteria that produce extended-spectrum beta-lactamases have become more common. These beta-lactamase enzymes make many, if not all, of the penicillins and cephalosporins ineffective as therapy. Extended-spectrum beta-lactamase—producing E. coli are highly resistant to an array of antibiotics, and infections by these strains are difficult to treat. In many instances, only two oral antibiotics and a very limited group of intravenous antibiotics remain effective. In 2009, a gene called New Delhi metallo-beta-lactamase (shortened as NDM-1) that even gives resistance to intravenous antibiotic carbapenem was discovered in India and Pakistan in E. coli bacteria.

Due to the severe nature of E. coli infection and the potential for lethality, it is necessary to develop alternative treatments for E. coli infection and for removal of E. coli bacteria from water and food. Thus, a method of removing Escherichia coli (E. coli) bacteria from an aqueous solution solving the aforementioned problems is desired.

### SUMMARY OF THE INVENTION

The method of removing Escherichia coli (E. coli) bacteria from an aqueous solution includes the step of mixing multi-walled carbon nanotubes into an aqueous solution containing E. coli bacteria. The multi-walled carbon nanotubes have an antimicrobial effect against the E. coli bacteria. The multi-walled carbon nanotubes may be mixed into the aqueous solution at a concentration of approximately 0.002 g of multi-walled carbon nanotubes per 100 ml of the aqueous solution. In order to enhance antimicrobial activity, the multi-walled carbon nanotubes in the solution, following the step of mixing, may be treated with microwave radiation, thus generating heat to further destroy the bacteria in the solution. In order to further enhance antimicrobial activity, the multi-walled carbon nanotubes may be functionalized with a carboxylic (COOH) group, functionalized with a phenol (C₅H₅OH) group, functionalized with a C18 group, such as 1-octadecanol (C₁₈H₃₈O), or may be impregnated with silver nanoparticles.

These and other features of the present invention will become readily apparent upon further review of the following specification and drawings.

### BRIEF DESCRIPTION OF THE DRAWINGS

FIG. 1 illustrates modification of multi-walled carbon nanotubes to produce multi-walled carbon nanotubes with carboxyl groups attached to the surface of the nanotubes.

FIG. 2 illustrates esterification of the modified multi-walled carbon nanotubes of FIG. 1.

FIG. 3 is a graph showing Fourier transform infrared spectral transmittance plots as functions of wavenumber for unmodified multi-walled carbon nanotubes, the modified multi-walled carbon nanotubes of FIG. 1, multi-walled carbon nanotubes further modified with an octadecanoate, and multi-walled carbon nanotubes further modified with a phenyl ester.

Similar reference characters denote corresponding features consistently throughout the attached drawings.

### DETAILED DESCRIPTION OF THE PREFERRED EMBODIMENTS

As will be described in detail below, the method of removing Escherichia coli (E. coli) bacteria from an aqueous solution includes the step of mixing multi-walled carbon nanotubes into an aqueous solution containing E. coli bacteria, The multi-walled carbon nanotubes have an antimicrobial effect against the E. coli bacteria. The multi-walled carbon nanotubes are preferably mixed into the aqueous solution at a concentration of approximately 0.002 g of multi-walled carbon nanotubes per 100 ml of the aqueous solution.

In order to enhance antimicrobial activity, the multi-walled carbon nanotubes in the solution, following the step of mixing, may be treated with microwave radiation, thus generating heat to further destroy the bacteria in the solution. The microwave radiation is preferably applied for at least five seconds, and more preferably is applied for approximately ten seconds.

In order to further enhance antimicrobial activity, the multi-walled carbon nanotubes may be functionalized with a carboxylic (COOH) group, functionalized with a phenol (C₅H₅OH) group, functionalized with a C18 group, such as 1-octadecanol (C₁₈H₃₈O), or may be impregnated with silver nanoparticles.

### Example

Multi-walled carbon nanotubes (MWCNTs) were purchased from Nanostructured & Amorphous Materials, Inc. of Houston, Tex. The purity of the MWCNTs was greater than 95%. The nanotubes had outer and inner diameters of approximately 10-20 nm and 5-10 nm, respectively. The length of each MWCNT was approximately 10-30 μm. A 300 ml solution of concentrated nitric acid (69% AnalaR Normapur® analytical reagent) was added to 2 g of the MWCNTs. The mixture was refluxed for 48 hours at 120° C. After cooling to room temperature, the reaction mixture was diluted with 500 ml of de-ionized water and then vacuum-filtered through a filter paper with 3 μm porosity. This washing operation was repeated until the pH became the same as that of de-ionized water, and was followed by drying in a vacuum oven at 100° C.

These conditions led to the removal of catalysts from the MWCNTs and opened both the tube caps, and also formed holes in the sidewalls of the nanotubes, followed by oxidative etching along the walls of the nanotubes with the concomitant release of carbon dioxide. This relatively non-vigorous treatment minimized the shortening of the tubes, so that the chemical modification was mostly limited to the opening of the tube caps and the formation of functional groups at defect sites along the sidewalls.

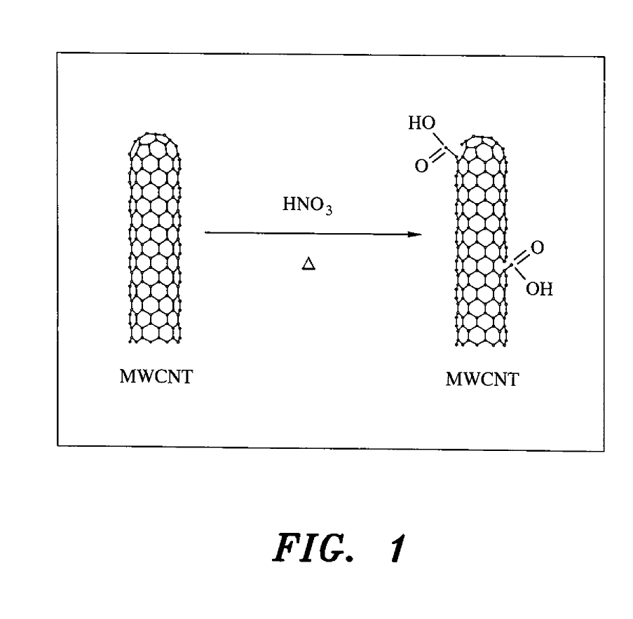

The final products were nanotube fragments whose ends and sidewalls were functionalized with various oxygen containing groups, carboxyl groups being prominent in the formation. FIG. 1 illustrates the chemical modification of the MWCNTs through thermal oxidation to produce the MWCNTs functionalized with carboxyl groups. Further, the percentage of carboxylic functions on the oxidized MWCNT surface did not exceed 4% in the most optimal cases, which corresponds to the percentage of MWCNT structural defects.

Fischer esterification (refluxing a carboxylic acid and an alcohol in the presence of an acid catalyst to produce an ester) is an equilibrium reaction. In order to shift the equilibrium to favor the production of esters, it is customary to use an excess of one of the reactants, typically either the alcohol or the acid. In the present reactions, an excess of the phenol (Aldrich, 98% purity) and 1-octadecanol (Merck, 97% purity) were used because they are cheaper and easier to remove than the MWCNTs. An alternative method of driving the reaction toward its products is the removal of one of the products as it forms. Water formed in this reaction was removed by evaporation during the reaction.

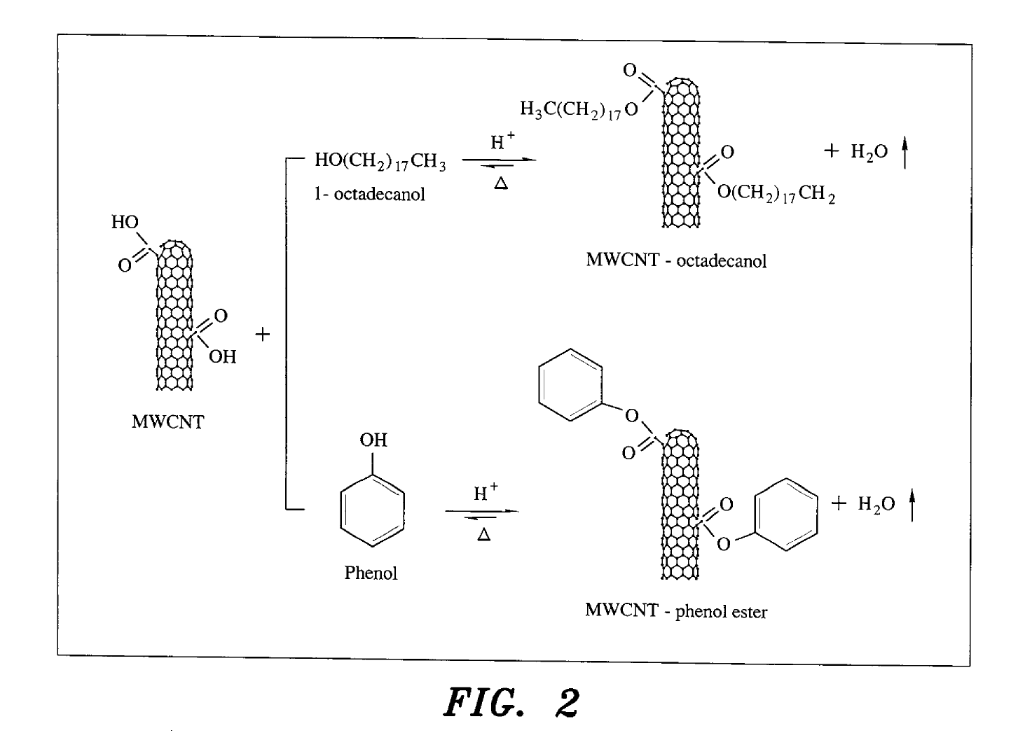

The oxidatively introduced carboxyl groups represent useful sites for further modifications, as they enable the covalent coupling of molecules through the creation of esters, as illustrated in FIG. 2. In a 250 ml beaker, 10 g of the material was melted on a hotplate at 90° C., and 1 g of MWCNTs was added. The mixture was stirred for 10 minutes, and then a few drops of sulfuric acid (as a catalyst) were added. After addition of the catalyst, the reaction remained on the hotplate and was stirred for two hours.

After completion of the reaction, the mixture was poured into 250 ml of benzene and vacuum-filtered through filter paper with 3 μm porosity. This washing operation was repeated five times, and was followed by washing with petroleum ether three times and with THF three times. The product was then washed with de-ionized water and acetone a few times, and then the produced functionalized MWCNT material was dried in a vacuum oven at 90° C.

The above particularly involves the activation of the carbonyl group by protonation of the carbonyl oxygen, nucleophilic addition to the protonated carbonyl to form a tetrahedral intermediate, and elimination of water from the tetrahedral intermediate to restore the carbonyl group.

Fourier Transform Infrared Spectroscopy (FTIR) has shown a limited ability to probe the structure of MWCNTs. A factor that has hindered the advancement of FTIR as a tool for MWCNT analysis is the poor infrared transmittance of MWCNTs. A solution to this problem was found through the use of KBr preparations of MWCNT samples. Because of their blackbody characteristics, the MWCNTs have a strong absorbance, and often are unable to be distinguished from background noise, thus making it necessary to use a very weak concentration of the MWCNTs in a KBr powder. However, the greater vibrational freedom of attached polymeric species presents much more pronounced peaks, and are thus typically the focus of attention in FTIR results.

Despite this, with very careful sample preparation, some researchers have managed to elucidate peaks corresponding to surface-bound moieties, such as carboxylic acid groups at wavenumbers of 1791, 1203 and 1080 cm⁻¹. The spectra of samples were recorded by a Perkin-Elmer 16F PCFT-IR spectrometer. FTIR samples were prepared by grinding dry material into potassium bromide, adding approximately 0.03% wt. This very low concentration of MWCNTs was necessary due to the high absorption of the carbon nanotubes.

Silver nanoparticles were impregnated on the surface of the MWCNTs using a wet impregnation technique. Silver nitrate was used as a source of silver nanoparticles. The silver salt was dissolved in ethanol solution and a fixed quantity of MWCNTs was dispersed in another ethanol solution. The two solutions were mixed at two ratios (10% and 50% of silver) and sonicated by ultrasonic sonicator for 30 minutes to form a homogenous dispersion of the MWCNTs and silver salt. Finally, the samples were filtered and dried in a vacuum oven overnight. The dried samples were calcinated in a tubular furnace at 350° C. for 3 hours to produce silver nanoparticles on the surface of the MWCNTs.

Strain E. coli ATCC number 8739 (supplied by the King Fand University of Petroleum and Minerals Clinic) was used. The E. coli was grown overnight in a nutrient broth at 37° C. on a rotary shaker (at 160 rpm). Aliquots of the preculture were inoculated into a fresh medium and incubated in the same conditions to an absorbance at 600 nm of 0.50. Cells were harvested by centrifugation at 4000 g for 10 min at 4° C., then washed twice with a sterile 0.9% NaCl solution at 4° C. and re-suspended in different carbon nanomaterials (MWCNTs, MWCNTs-COOH, MWCNTs-Phenol, MWCNTs-C18, MWCNTs-10% Ag, MWCNTs-50% Ag) solution to a concentration of 2×10⁷ CFU/ml.

All carbon nanomaterial was sonicated before mixing with the bacterial solution. For each type of carbon nanomaterial used, the mixture of carbon nanomaterial and bacteria was tested, both with and without exposure to microwave radiation for 0, 5, and 10 seconds. Cultured bacteria (tested bacteria with different carbon nanomaterials, both with and without microwave treatment) were analyzed by plating on nutrient agar plates after serial dilution in 0.9% saline solution. Colonies were counted after 48 hours of incubation at 37° C. Control experiments were carried out in parallel with each experiment performed for the particular MWCNT material tested; i.e., testing of E. coli in MWCNT materials without microwave radiation treatment.

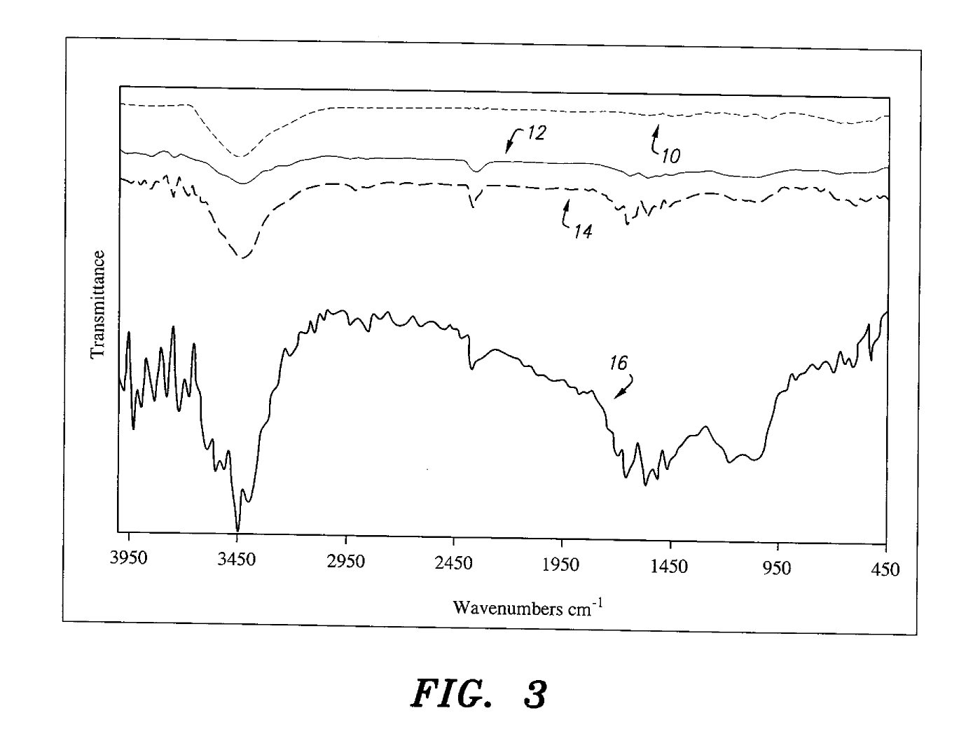

The untreated and unmodified MWCNTs showed a very weak peak at around 1635 cm⁻¹, as shown by line 10 in FIG. 3. This is due to the oscillation of carboxylic groups. This peak moves to 1730 cm⁻¹ (associated with the stretch mode of carboxylic groups) observed in the infrared (IR) spectrum of the acid-treated MMWNTs (shown as line 12 in FIG. 3). This indicates that carboxylic groups were formed along with a C═O liaison of the carboxylic acid function due to the oxidation of some carbon atoms on the surface of the MWCNTs by the nitric acid.

The IR spectra of oxidized MWCNTs show four major peaks at 3750, 3450, 2370 and 1562 cm⁻¹. The peak at 3750 cm⁻1 is attributed to free hydroxyl groups. The peak at 3445 cm⁻¹ is attributed to O—H stretch from carboxylic groups (O═C—OH and C—OH), while the peak at 2364 cm⁻¹ is associated with OH stretch from strong H-bond-COOH. The peak at 1565 cm⁻¹ is related to the carboxylate anion stretch mode. It should be noted that the unmodified MWCNTs were purified and a part of the catalytic metallic nanoparticles were possibly eliminated during the purification process, cutting the nanotube cap. Thus, the presence of carboxylic groups in these commercial MWCNTs was expected. Moreover, it should be noted that there is no significant difference between the spectra of the samples before and after the HNO₃ treatment.

The peak at 1635 cm⁻¹ is associated with the stretching of the carbon nanotube backbone. Increased strength of the signal at 1165 cm⁻¹ is attributed to C—O stretching in the same functionalities. The peaks around 2877 and 2933 cm⁻¹ correspond to the H—C stretch modes of H—C═O in the carboxylic group.

In line 14 of FIG. 3, the MWCNT—COO(CH₂)₁₇CH₃ showed IR absorptions at 2924 cm⁻¹ and 2872 cm⁻¹ (the C—H stretch modes), which is indicative of the presence of a long-chain alkyl molecule of octadecanol of the alkyl chain, with a peak at 1701 cm⁻¹ (from C═O stretch of the ester). The peak at 1560 cm⁻¹ is from the C═C stretch of the MWCNTs, and the peak at 1461 cm⁻¹ is attributed to the C—H bend of the alkyl chain. The peak at 1108 cm⁻¹ is due to the C—O stretch of the ester group. Many of these absorptions are known for functionalized single-walled carbon nanotubes. Further, the spectra for MWCNT-octadecanoate exhibited the typical bands of the —CH₂ rocking at 760 cm⁻¹. It should be noted that all lines in FIG. 3 show peaks between 1300 and 1100 cm⁻¹, which are attributed to the C—C stretch bonds.

As can be seen in line 16 of FIG. 3, the spectra correspond to the phenyl structure, as follows. The peaks between 3000 and 3140 cm⁻¹ correspond to the bands of stretching of the group C—H of the aromatic ring; the peaks between 1600 and 2000 cm⁻¹ are attributed to the overtones of the phenyl ring substitution; the peaks at 685 and 890 cm⁻¹ represent the bands of phenyl ring substitution; and the peaks at 1565, 1505 and 1460 cm⁻¹ are attributed to the bands of stretching of the group C═C of the aromatic ring.

Table 1 below illustrates the percentage of E. coli removal in aqueous solution from the addition of unmodified multi-walled carbon nanotubes, multi-walled carbon nanotubes functionalized with a carboxylic (COOH) group, multi-walled carbon nanotubes functionalized with a phenol (C₅H₅OH) group, multi-walled carbon nanotubes functionalized with a C18 group (1-octadecanol (C₁₈H₃₈O)), and multi-walled carbon nanotubes impregnated with silver nanoparticles (with silver nanoparticles added at both 10 wt % and 50 wt %).

TABLE 1
E. coli removal without microwave heating
Number of E. Coli Cells

| Type of MWCNT | | | |
|---|---|---|---|
| added to control | | After addition of | % of E. coli |
| sample | Control sample | MWCNTs | removal |
| MWCNT | 3.70 × 10⁷ | 3.50 × 10⁷ | 5 |
| MWCNT-COOH | 3.70 × 10⁷ | 3.60 × 10⁷ | 3 |
| MWCNT-C18 | 3.50 × 10⁷ | 3.40 × 10⁷ | 3 |
| MWCNT-Ag (10 wt | 2.20 × 10⁷ | 1.90 × 10⁷ | 14 |
| %) | | | |
| MWCNT-Ag (50 wt | 2.20 × 10⁷ | 1.70 × 10⁷ | 23 |
| %) | | | |
| MWCNT-Phenol | 2.30 × 10⁷ | 1.60 × 10⁷ | 30 |

As shown in Table 1, the results indicate that the percentage of E. Coli bacteria removal for MWCNTs, MWCNTs-COOH and MWCNTs-C18 is relatively small (3-5 wt %). Although MWCNTs and MWCNTs-C18 have similar properties due to their similar C—C bonding, MWCNTs-COOH provide different properties due to the carboxylic group. On the other hand, the percentage of E. Coli bacteria removal for MWCNTs-Ag is increased due to the presence of the Ag nanoparticles. Increasing the amount of Ag nanoparticles from 10 wt % to 50 wt % almost doubles the percentage of bacterial removal.

The highest removal rate of E. coli bacteria was shown with MWCNTs functionalized with phenol functional groups. From Table 1, it is clearly shown that the MWCNTs-phenol removed almost 30% of E. coli bacteria from solution, even without the application of microwave radiation.

Table 2 below illustrates the percentage of E. coli removal in aqueous solution from the addition of unmodified multi-walled carbon nanotubes, multi-walled carbon nanotubes functionalized with a carboxylic (COOH) group, multi-walled carbon nanotubes functionalized with a phenol (C₅H₅OH) group, multi-walled carbon nanotubes functionalized with a C18 group (1-octadecanol (C₁₈H₃₈O)), and multi-walled carbon nanotubes impregnated with silver nanoparticles (with silver nanoparticles added at both 10 wt % and 50 wt %) with the added step of microwave treatment, both for a duration of 5 seconds and for a duration of 10 seconds.

TABLE 2
E. coli removal with microwave heating

| Type of MWCNT | Number of E. Coli | % of E. coli | % of E. coli |
|---|---|---|---|
| added to control | Cells in | removal | removal |
| sample | control sample | at 5 sec. | at 10 sec. |
| No MWCNTs | 3.70 × 10⁷ | 8.1 | 59.5 |
| added | | | |
| MWCNT | 3.70 × 10⁷ | 40.5 | 77.0 |
| MWCNT-COOH | 3.70 × 10⁷ | 18.9 | 64.9 |
| MWCNT-C18 | 3.50 × 10⁷ | 37.1 | 97.1 |
| MWCNT-Ag (10 wt | 2.20 × 10⁷ | 59.1 | 100 |
| %) | | | |
| MWCNT-Ag (50 wt | 2.20 × 10⁷ | 94.1 | 100 |
| %) | | | |
| MWCNT-Phenol | 2.30 × 10⁷ | 32.6 | 76.1 |

As shown in Table 2, the heat generated from the microwave treatment removed 8% of E. coli from water after 5 seconds of exposure and up to 60% after 10 seconds. The percentage removal of E. coli bacteria after 5 seconds of microwave heating increased from 8% to 40% through the addition of a relatively small amount of MWCNTs to the water (0.2 g of MWCNTs/100 ml). The MWCNTs absorb the microwave heat and convert it to local heating within 5 seconds. A higher removal rate of E. coli bacteria (77%) was achieved after 10 seconds of microwave treatment.

On the other hand, the results indicate that the percentage of E. coli bacteria removal using MWCNTs modified with a carboxylic group (i.e., MWCNTs-COOH) in water decreased drastically from 40% to about 19% after 5 seconds of microwave exposure. The reason for this behavior is that the COON group changed the thermal properties of the MWCNTs by forming (on the surfaces of the MWCNTs) chains of C—O—O—H, which are not thermally conductive. Increasing the exposure time to 10 seconds gave the same result. The behavior of MWCNTs modified with phenol, in terms of removal efficiency of the bacteria, after 5 seconds and 10 seconds of microwave exposure is similar to that of unmodified MWCNTs.

Similar results were obtained using MWCNTs modified with a C18 group after 5 seconds, while a high removal rate of E. coli bacteria was achieved after 10 seconds due to multiple chains of C18 (C—C bonds), which increased the absorption rate of the microwave heat.

For the MWCNTs impregnated with Ag nanoparticles, the percentage of E. coli bacteria removal after 5 seconds of microwave exposure was the highest, compared to the above mentioned materials, indicating that the absorption of microwave heat is the highest due to the presence of Ag nanoparticles. Increasing the amount of impregnated Ag nanoparticles from 10 wt % to 50 wt % drastically enhanced the percentage of bacterial removal from 60% to 94%. For 10 seconds of microwave exposure, all E. coli bacteria was removed from water (100% removal rate) for both Ag concentrations.

It is to be understood that the present invention is not limited to the embodiments described above, but encompasses any and all embodiments within the scope of the following claims.

We claim:

1. A method of removing E. coli bacteria from an aqueous solution, comprising the step of mixing multi-walled carbon nanotubes into an aqueous solution containing E. coli bacteria, wherein the multi-walled carbon nanotubes have an antimicrobial effect against the E. coli bacteria.

2. The method of removing E. coli bacteria from an aqueous solution as recited in claim 1, wherein multi-walled carbon nanotubes are mixed into the aqueous solution at a concentration of about 0.002 g of multi-walled carbon nanotubes per 100 ml of the aqueous solution.

3. The method of removing E. coli bacteria from an aqueous solution as recited in claim 2, further comprising the step of treating multi-walled carbon nanotubes and the aqueous solution with microwave radiation following the step of mixing.

4. The method of removing E. coli bacteria from an aqueous solution as recited in claim 3, wherein multi-walled carbon nanotubes and the aqueous solution are treated with the microwave radiation for about 10 seconds.

5. A method of removing E. coli bacteria from an aqueous solution, comprising the step of mixing functionalized multi-walled carbon nanotubes into an aqueous solution containing E. coli bacteria, wherein the functionalized multi-walled carbon nanotubes have an antimicrobial effect against the E. coli bacteria.

6. The method of removing E. coli bacteria from an aqueous solution as recited in claim 5, wherein the functionalized multi-walled carbon nanotubes are mixed into the aqueous solution at a concentration of about 0.002 g of functionalized multi-walled carbon nanotubes per 100 ml of the aqueous solution.

7. The method of removing E. coli bacteria from an aqueous solution as recited in claim 6, wherein the functionalized multi-walled carbon nanotubes are functionalized with a carboxylic (COOH) group.

8. The method of removing E. coli bacteria from an aqueous solution as recited in claim 7, further comprising the step of treating the functionalized multi-walled carbon nanotubes and the aqueous solution with microwave radiation following the step of mixing.

9. The method of removing E. coli bacteria from an aqueous solution as recited in claim 8, wherein the functionalized multi-walled carbon nanotubes and the aqueous solution are treated with the microwave radiation for about 10 seconds.

10. The method of removing E. coli bacteria from an aqueous solution as recited in claim 7, wherein the functionalized multi-walled carbon nanotubes are functionalized with a phenol (C₅H₅OH) group.

11. The method of removing E. coli bacteria from an aqueous solution as recited in claim 10, further comprising the step of treating the functionalized multi-walled carbon nanotubes and the aqueous solution with microwave radiation following the step of mixing.

12. The method of removing E. coli bacteria from an aqueous solution as recited in claim 11, wherein the functionalized multi-walled carbon nanotubes and the aqueous solution are treated with the microwave radiation for about 10 seconds.

13. The method of removing E. coli bacteria from an aqueous solution as recited in claim 7, wherein the functionalized multi-walled carbon nanotubes are functionalized with a C18 group.

14. The method of removing E. coli bacteria from an aqueous solution as recited in claim 13, wherein the functionalized multi-walled carbon nanotubes are functionalized with 1-octadecanol (C₁₈H₃₈O).

15. The method of removing E. coli bacteria from an aqueous solution as recited in claim 14, further comprising the step of treating the functionalized multi-walled carbon nanotubes and the aqueous solution with microwave radiation following the step of mixing.

16. The method of removing E. coli bacteria from an aqueous solution as recited in claim 15, wherein the functionalized multi-walled carbon nanotubes and the aqueous solution are treated with the microwave radiation for approximately 10 seconds.

17. A method of removing E. coli bacteria from an aqueous solution, comprising the step of mixing multi-walled carbon nanotubes impregnated with silver nanoparticles into an aqueous solution containing E. coli bacteria, wherein the multi-walled carbon nanotubes impregnated with silver nanoparticles have an antimicrobial effect against the E. coli bacteria.

18. The method of removing E. coli bacteria from an aqueous solution as recited in claim 17, wherein the multi-walled carbon nanotubes impregnated with silver nanoparticles are mixed into the aqueous solution at a concentration of about 0.002 g of multi-walled carbon nanotubes impregnated with silver nanoparticles per 100 ml of the aqueous solution.

19. The method of removing E. coli bacteria from an aqueous solution as recited in claim 18, further comprising the step of treating the multi-walled carbon nanotubes impregnated with silver nanoparticles and the aqueous solution with microwave radiation following the step of mixing.

20. The method of removing E. coli bacteria from an aqueous solution as recited in claim 19, wherein the multi-walled carbon nanotubes impregnated with silver nanoparticles and the aqueous solution are treated with the microwave radiation for about 10 seconds.
